(19) United States
(12) Patent Application Publication
(10) Pub. No.: US 2010/0279338 A1
(43) Pub. Date: Nov. 4, 2010

(54) CELL CARRIER CODING

(76) Inventors: Asaf Halamish, Pardes Chana (IL); Michael Sister, Holon (IL); Asaf Ben-Arye, Zichron Yaakov (IL)

Correspondence Address: Daniel Feigelson, Lev Hamada Building, Second Floor, 7 Oppenheimer St., Rehovot, IL

(21) Appl. No.: 12/678,834

(22) PCT Filed: Sep. 21, 2008
(86) PCT No.: PCT/IL08/01258

§ 371 Date: Jun. 29, 2010

Related U.S. Application Data
(60) Provisional application No. 60/973,464, filed on Sep. 19, 2007.

(51) Int. Cl. C12Q 1/04; C12M 1/34

(52) U.S. Cl. 435/34; 435/307.1

(57) ABSTRACT

There are provided cell carrier devices for use in cell observation systems, enabling determination of the location of the region under observation by virtue of coding information present in the field of view of the viewing device, even at high magnification. Other embodiments are also disclosed.

### FIELD OF THE INVENTION

The present invention relates to the field of cell carriers for the observation of trapped cells, especially cell carriers divided into separate regions, each of those regions incorporating orientation and location identification that can be readily acquired by the cell observation system.

### BACKGROUND OF THE INVENTION

Carriers for the analysis of a plurality of individual living cells are known in the art. For example, U.S. Pat. Nos. 4,729,949, 4,772,540, 5,272,081, 5,310,674, 5,506,141, 6,495,340, and co-pending, commonly-assigned PCT patent application PCT/IB2007/000545, the contents of each of which are incorporated herein by reference, each in its entirety, describe cell carriers comprising grids, each having a plurality of holes which are generally open at both faces of the cell carrier and which are shaped and sized to enable each hole to contain one or more living cells. The grids enable the cells to be observed using visible or ultraviolet light and conventional optics. Cell carriers having holes therethrough, are generally referred to as “hole-type cell carriers”. Because the holes in a hole-type cell carrier are in a predefined arrangement, each hole may in principle be given an address, and thus hole-type cell carriers facilitate the repeated viewing of a cell or cells contained therein through a microscope or other device, by directing the microscope or other device to return to the desired address or addresses.

Similarly, cell carriers which utilize grids or other arrangements of wells sized to hold individual cells, which are open at only one face of the cell carrier, are known in the art, see e.g. U.S. patent publications nos. 2006/0057557, 2005/0014201, 2005/0064524, 2006/0154233, and 2006/0240548, and PCT publications WO 2005/007796, WO 2006/043267, WO 2006/021959 and WO 2007/052245, the entire contents of each of which are incorporated herein by reference. Like hole-type cell carriers, the wells in a well-type cell carrier have a predefined arrangement, and thus each well may in principle be given an address to facilitate the repeated viewing of a cell or cells contained therein through a microscope or other viewing device. It will be appreciated that although in some well-type cell carriers, the wells are adapted to contain a plurality of living cells, generally up to about 10 cells, other well-type cell carriers may be constructed and adapted to contain an individual living cell.

Hole-type cell carriers and well-type cell carriers which are adapted to hold one or more living cells per location are generally referred to collectively as cell carriers having cell-capturing depressions, “depressions” referring to either the holes or the wells, and “cell-capturing” referring to the property that each of the depressions is sized to hold one or more cells of a particular type.

One difficulty with such cell carriers is ascertaining the address of a depression that is being viewed. One prior art solution is to incorporate a reference point at a predefined position of the cell carrier, for instance, at one corner of the cell carrier, and then to move the cell carrier in a predetermined direction and by a predetermined distance to enable viewing of a desired position whose co-ordinates relative to the reference point of the cell carrier are known. A disadvantage of such a prior art method is that under the high magnification used with such cell observation systems, only a portion of the grid is generally seen in any image. Therefore, if only a single reference point is used at a predefined position of the cell carrier, most of the areas of the cell carrier viewed at high magnification will not contain any information about the location being viewed. Consequently, the viewing device has to be switched to a low magnification state in order to acquire the reference point position, and the area desired to be viewed then has to be reached by reliance on relative motion between the viewing device and the sample stage, before or after return to the high magnification state. This method may be disadvantageous, both because the table motion takes time, since every new location must be referred to the marked origin point, and because it may be inaccurate because of the need to traverse a large number of locations to arrive exactly on target at the desired location. This latter disadvantage may apply whether each table motion is referred back to the origin for each motion, or whether each motion is made additive to the previous position, in which case any error may be accumulative. Furthermore, an experienced cell sample analyst can readily learn to reposition the cell carrier manually, even under high magnification, very close to the region to be viewed, without recourse to the motion system. If no location information is available in the high magnification images, then the exact location of the depressions in the region cannot be determined without stepping back down to low magnification, and the advantage of the operator's skill is thus lost.

It would be useful to be able to have cell carriers, both hole-type and well-type, for use in a viewing system at high magnification, which enable the determination of which portion of a cell carrier is being observed, in an accurate and rapid manner, while generally relying only on information contained within the field of view of the viewing device used to observe the cells in the cell carrier. Use of such a cell carrier should ensure that any image input to the image processing routine will contain address information without the need to reduce magnification, or to move the stage.

### SUMMARY OF THE INVENTION

The present disclosure describes new cell carrier devices, and methods for their use in cell carrier observation systems, which enable the location of the region under observation to be uniquely determined, even at high magnification, by virtue of coding information present in the field of view of the viewing device. This is achieved by dividing up the entire field of cell capturing locations into a number of separate regions, each separate region being encoded with information which the system uses to define the location within the complete cell carrier of that particular region. The coding information may also define the orientation of that particular region relative to a predefined orientation of the cell carrier. The coding information should be contained in predetermined areas of each region, and the image processing unit of a viewing system designed to use such cell carriers should be adapted to detect the presence of the coding information in an image, and from its position, to define the limits of the region which that coding information is associated with. This means that the regions into which the entire cell carrier is divided do not need to have physically separate boundaries, but rather are defined within the continuum of call capturing locations, by the positions of the encoding information and the predetermined knowledge of the extent of each region around the positions of that encoding information. One practical method of providing the coding information is by marking some of the cell capturing locations according to a predetermined identifying pattern, as will be expounded in more detail hereinbelow.

The regions should be of such a size that even under the high magnification generally used to view the cells, coding information appears in any image of the cell carrier. In this respect, although no numerical values are given in this application for the term “high magnification”, the term is understood to mean a level of working magnification of the order generally used to view the cell types for which the cell carrier is provided, which is generally a significantly higher magnification that that which enables the whole of the cell carrier to be viewed.

Conversely, the predetermined areas of each region in which coding information is contained, should be small enough that a complete code becomes visible in an image frame acquired at the working magnification of the system. There are two alternative or additive modes of operation of the system with respect to these limitations. According to one alternative, it is sufficient for any image frame to acquire just a single part of the entire coding information, such as a single mark, and using that single part of the information, the motion system is programmed to move the cell carrier such that all of the coding information is visible, and preferably in the center of the image frame. Such an implementation can also be performed manually using visual observation by the operator. According to another alternative, the size of each region, or more exactly, the size of the coding area in relation to each region is made such that the entire coding information can be found in any image frame obtained, and the image processing unit can then ascertain the above mentioned information immediately without the need to move the cell carrier. It is appreciated that these two alternative modes of operation are complementary, and are dependent on the maximum magnification with which it is desired to use such cell carriers. For a given ratio of coding information area to size of region, the method in which acquisition of a single part of the coding information is sufficient can obviously be used initially at a higher magnification level than the method in which the entire code is acquired in any image, though ultimately, both alternative modes of operation require that the entire code be contained within one image frame, with or without motion to achieve that object. Once the entire code is acquired, and preferably is centered in the image, the image processing unit can define the “boundaries” of that region by knowledge of the extent of the region from the area of the coding information, can ascertain the identity of the location of that region within the entire cell carrier, and can ascribe unique addresses to each cell capturing location within that region.

Use of such cell carrier devices obviates the need to switch the viewing system to low magnification in order to acquire location information in its field of view. By this means, it is immediately known where the area being viewed is situated within the very large number of cell capturing depression locations, and that location information can be used to return rapidly to that region to review a cell depression previously viewed.

As is generally done in such cell carrier observation systems, each cell capturing depression is provided with its own unique address, and once the automatic image processing unit of the system has deciphered, from the arrangement of the indicia seen in the image being processed, the location (and orientation) of the region within the entire cell carrier, each depression within that image can then be referred to by its unique location address. In the case of an automated system, the system control generally instructs the motion system to navigate to the desired location according to the detected coding information. When visually directed motion may be used, the operator can use the location information in any field of view to position the cell carrier on the stage with good precision.

Once the location information is determined, the motion system can be used to rapidly move to the specific region desired, and since that region is generally significantly smaller in area than that of the entire cell carrier, and therefore has a significantly smaller number of locations within its area, the image processing, computing and motion generating facilities required to navigate within that region to reach the desired location are significantly less than those of a prior art system, where the corresponding facilities may have to cover the entire cell carrier.

According to one exemplary implementation of such cell carriers, the regions are encoded by means of indicia markings on or in individual cell capturing locations, with the encoding information regarding the location and orientation of the region being defined by the location and number of indicia within a small predefined area of each region. One exemplary implementation may use one set of indicia to define orientation, and the location of one or more additional indicia to define the identifying location of the region within the cell carrier. The indicia may be grouped together in one limited area of the region, such as in the central area, or, in order not to limit the use of one concentrated area of the region, they may be spread out in predefined positions over the entire area of the region.

For those systems which use optically transparent cell carriers, the indicia markings may be implemented by means of opaque spots on the cell capturing locations. A common method of viewing the cell capturing locations is by means of visible or ultra-violet light, though it is to be understood that the systems and devices described herein are not meant to be limited to these illumination methods, but that other wavelengths can also be used, without departing from the scope of this disclosure. Since the application of such opaque spots may require an additional manufacturing step, they may be alternatively implemented by the simple procedure of manufacturing the cell carriers with an optical diffusing layer in the cell capturing depression locations to be marked, or with missing cell capturing depressions at the appropriate encoded indicia locations, such that the optical transmission of the cell carrier is different at those locations. For those systems which use optically opaque cell carriers, the indicia markings may be implemented by means of closing off cell capturing locations, which would otherwise be clear holes.

Although the various implementations of the devices in this disclosure have been described in terms of encoding indicia marked on the cell capturing depressions themselves, it is to be understood that any kind of detectable marking which can be uniquely associated with a specific depression may be used. Since the cell carriers are generally made with maximum depression density possible, the typical space available between the cell capturing depressions may be limited, For instance, a typical cell carrier may have 15 micron holes, and the spacing between holes may be as small as 20 microns, such that only a 5 micron space is available for marking between holes. Consequently, marking the locations between depressions may not be simple, but where feasible, may be performed to define a coded depression position without affecting the functionality of the depression itself.

According to further suggested examples of such cell carrier devices, the encoding information regarding the location of a region can be determined by use of either a single index, or by means of more than one index. The latter alternative may be particularly useful for situations where very large numbers of cell capturing locations are required, which it is then advantageous to divide up into a larger number of regions, so that each region should remain of manageable size, or when it is desired not to take up too many of the cell capturing locations with encoding indicia.

When such large numbers of cell capturing locations are required, according to another suggested exemplary implementation, several separate grids of cell capturing depressions, each grid being split into its own set of separate regions, may be mounted onto one cell carrier, and the location encoding indicia of a particular region can then provide information about both the location of the region within the grid, and about the location of that specific grid on the complete cell carrier. This information may be in addition to the orientation information also available from the indicia arrangement. By this means, rapid location of a desired cell capturing location can be achieved on cell carriers with very large numbers of cell capturing depressions. It is to be understood that the grids can be either separate physical grids on the cell carrier, or simply “super regions” on a single physically large grid, each “super-region” then being split up into its previously described regions. Each encoded location address then defines not only the region within a specific “super-region” on the cell carrier, but also the location of that “super-region” within the entire cell carrier grid structure.

According to one aspect of the present invention, and as described in this disclosure, there is provided a cell carrier comprising an array of cell-capturing depressions, the array being organized into a plurality of regions of cell-capturing depressions, each of said regions being encoded with a plurality of indicia associated with selected ones of said cell capturing depressions within that region, and which uniquely identify the region and indicate the orientation of the region relative to the cell carrier.

According to further exemplary implementations of the cell carriers described in this disclosure, there is provided a cell carrier comprising a plurality of cell-capturing depressions organized into a plurality of regions, some of the cell-capturing depressions of each of the regions being marked, wherein the spatial arrangement of the marked cell-capturing depressions in a region identifies the location of the region within the cell carrier. The spatial arrangement of the marked cell-capturing depressions within a region may further provide an indication of the orientation of that region. In such cell carriers, the marked cell-capturing depressions may be arranged in predetermined locations in a section of the regions, such that the location of the region within the cell carrier is identified by inspection of the predetermined locations in the section of the region.

The predetermined locations within a region of the above mentioned marked cell-capturing depressions may advantageously be arranged such that at least one marked depression is visible in an image of the region taken at a magnification significantly higher than that which enables the entire cell carrier to be imaged. Alternatively, the predetermined locations may be arranged such that all of the marked cell-capturing depressions of a specific region are visible in an image of the region taken at a magnification significantly higher than that which enables the entire cell carrier to be imaged.

In alternative implementations of any of the above-described cell carriers, the marks of the marked cell-capturing depressions may possess a detectable property that distinguishes a marked cell-capturing depression from other cell-capturing depressions in the cell carrier. This detectable property may be selected from the group consisting of (a) transmission of electromagnetic radiation of a particular wavelength or range of wavelengths, (b) absorption of electromagnetic radiation of a particular wavelength or range of wavelengths, (c) fluorescence at a particular wavelength in response to stimulation at a different particular wavelength, (d) reflection of electromagnetic radiation of a particular wavelength or range of wavelengths, and (e) optical interference of electromagnetic radiation of a particular wavelength or range of wavelengths. In cases where the detectable property is absorption of electromagnetic radiation in the visible spectrum, the marking should have decreased optical transmission in comparison with other areas of the cell carrier. According to another exemplary implementation, the cell carrier may be constructed of a material which is substantially transparent to visible light, and the marking may then be substantially opaque to visible light.

Furthermore, in any of the above-described exemplary cell carriers, the marking of the cell-capturing depressions may be located either within the cell-capturing depression, or in the vicinity of the cell-capturing depression.

In a further exemplary cell carrier, the section of the regions containing the marked cell-capturing depressions may comprise a first set of marked cell-capturing depressions and a second set of unmarked cell-capturing depressions. In this case, the first set of marked cell-capturing depressions may be associated with four cell-capturing depression locations arranged in a quadrilateral pattern. Additionally, the location of some of the first set of marked cell-capturing depressions may define the spatial orientation of the region, and at least one other of the first set of marked cell-capturing depressions may define the location of the region in the cell carrier.

Still other example implementations involve a method for identifying the location of a region of a cell carrier under observation, comprising:

(i) providing a cell carrier comprising a plurality of cell-capturing depressions organized into a plurality of regions, some of the cell-capturing depressions of each of the regions being marked, the spatial arrangement of the marked cell-capturing depressions in a region identifying the location of that region within the cell carrier,
(ii) illuminating the cell carrier with a source,
(iii) detecting marked cell-capturing depressions in an image of the cell carrier, and
(iv) identifying the location of the region of the cell carrier from the spatial arrangement of the marked cell-capturing depressions.
This exemplary method may also comprise the step of detecting the orientation of the region from the spatial arrangement of the marked cell-capturing depressions.

In either of the above described methods, the image of the cell carrier may be obtained at a magnification significantly higher than that which enables the entire cell carrier to be imaged. These methods thus enable the identification of the location of the region without reduction in the magnification used to view cell capturing depressions in the cell carrier.

Furthermore, according to another exemplary method described in this disclosure, the location of a specific cell capturing depression under observation in a cell carrier can be identified by use of the above described methods of identifying the region of the cell carrier in which the specific cell capturing depression is located, and then of determining the location of the specific cell capturing depression within the region. An optional additional step of detecting the orientation of the region of the cell carrier in which the specific cell capturing depression is located can be performed before determining the location of the specific cell capturing depression within the region. In either of these exemplary methods, the step of determining the location of the specific cell capturing depression within the region may be performed by an image processing routine using the spatial arrangement of marked cell-capturing depressions and the known extent of a region around the spatial arrangement of marked cell-capturing depressions. In any of the above described example of methods described in this disclosure, return to the observation of a previously viewed cell in a cell-capturing depression may be enabled without the need to search for location identifying information at low magnification.

In any of the above described implementations of methods of the present application, the marking of the marked cell-capturing depressions may possess a detectable property that distinguishes a marked cell-capturing depression from other cell-capturing depressions in the cell carrier. This detectable property may be selected from the group consisting of (a) transmission of electromagnetic radiation of a particular wavelength or range of wavelengths, (b) absorption of electromagnetic radiation of a particular wavelength or range of wavelengths, (c) fluorescence at a particular wavelength in response to stimulation at a different particular wavelength, (d) reflection of electromagnetic radiation of a particular wavelength or range of wavelengths, and (e) optical interference of electromagnetic radiation of a particular wavelength or range of wavelengths.

In cases where the detectable property is absorption of electromagnetic radiation in the visible spectrum, the marking should have decreased optical transmission in comparison with other areas of the cell carrier. According to another exemplary implementation, the cell carrier may be constructed of a material which is substantially transparent to visible light, and the marking may then be substantially opaque to visible light.

Furthermore, in any of the above-described exemplary methods, the marking of a cell-capturing depression may be located either within the cell-capturing depression, or in the vicinity of the cell-capturing depression.

Throughout this disclosure, since the application is directed at methods of providing addresses to the depressions, and not to the contents of those depression—whether single cell or multiple cell—it is to be understood that any references to a cell in a depression, or to cells in a depression, or similarly phrased expressions, are meant to include either of those cases of cell carriers in which there can be more than one cell per depression, and those which can accommodate only an individual cell per depression.

### BRIEF DESCRIPTION OF THE DRAWINGS

The presently claimed invention will be understood and appreciated more fully from the following detailed description, taken in conjunction with the drawings in which:

FIG. 1 illustrates schematically an exemplary system which incorporates a cell carrier in accordance with some aspects of the present invention;

FIGS. 2A to 2C depict some details of a 2500 hole-type cell carrier, divided into 25 regions, and whose construction and operation is described in this disclosure;

FIG. 3A depicts schematically the central 16 cell-capturing depression locations of each region of the 25-region cell carrier shown in FIGS. 2A to 2C, while FIG. 3B depicts schematically the orientation-defining cell-capturing depression locations of the central 16 locations in any region of the cell carrier of FIG. 3A;

FIG. 4A depicts schematically the central 16 cell-capturing depression locations of several representative regions of a cell carrier having 36 regions of cell-capturing depressions, while FIG. 4B depicts schematically the orientation-defining cell-capturing depression locations of the central 16 locations in any region of the cell carrier of FIG. 4A;

FIGS. 5A to 5E depict schematically the central 25 cell-capturing depression locations of several representative regions of a cell carrier;

FIG. 6A depicts schematically a cell carrier incorporating four separate grid areas, while FIGS. 6B to 6D show schematically several 10×10 regions within a grid area of the cell carrier of FIG. 6A;

FIG. 7 illustrates schematically a cross sectional view of another exemplary cell carrier, in which the indicia marking at the relevant cell capturing location is achieved by the omission of the cell capturing depression itself;

FIG. 8A illustrates schematically a plan view of a section of an exemplary region of a cell carrier, where the indicia markings are achieved by use of an optical interference effect, while FIG. 8B shows a typical image obtained from the device shown in FIG. 8A; and

FIGS. 9A and 9B illustrate schematically other methods by which the coding markings can be detected; FIG. 9A shows reflective markings, while FIG. 9B shows fluorescent or phosphorescent markings.

### DETAILED DESCRIPTION

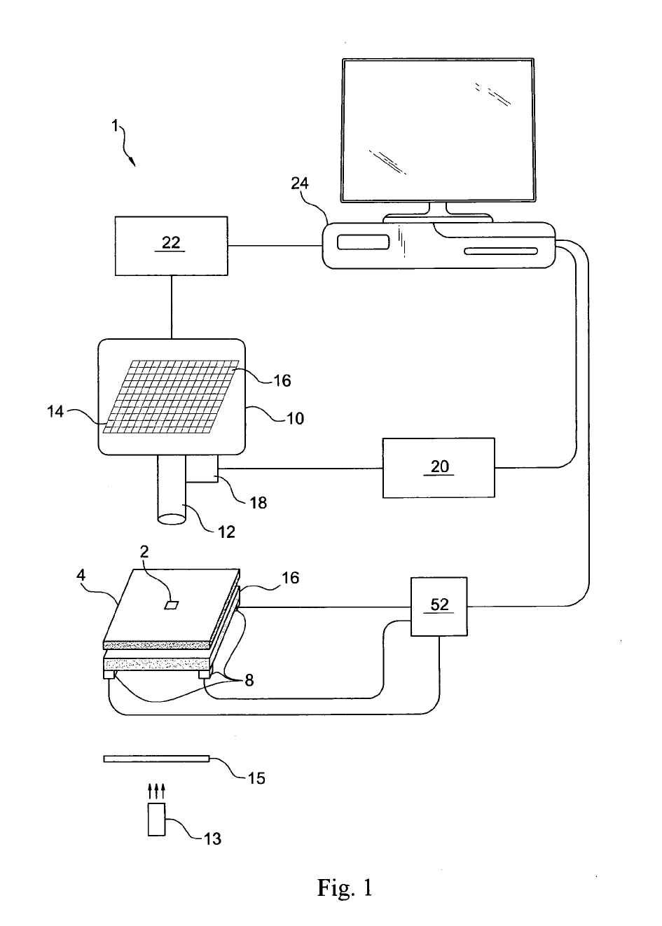

Reference is now made to FIG. 1, which illustrates schematically an exemplary system 1 which incorporates a cell carrier in accordance with some aspects of the present invention and which can be used to practice methods in accordance with other exemplary aspects of the present invention. In FIG. 1, a cell carrier 2 is shown located on a holder or support 4. Cell carrier 2 may be a hole-type cell carrier or a well-type cell carrier having wells constructed and operative to hold cells. Cell carrier 2 is affixed to holder/support 4, for example by use of adhesive, ultrasonic welding or mechanically, such as by way of clamps (not shown in FIG. 1), as appropriate. At least the portion of holder/support 4 above which cell carrier 2 rests should be transparent at the wavelengths of the radiation with which cell carrier 2 will be illuminated, and it will be appreciated that in some examples of the system, cell carrier 2 may rest above a hole formed in holder/support 4. Holder/support 4 may itself be mounted on a mount 6, which may be attached to a set of motors 8 capable of moving mount 6, and consequently holder/support 4, in very small increments, to enable observation of each well/hole, and thus each cell in each well/hole in cell carrier 2. The operation of motors 8—and thus the positioning of support/holder 4—may be controlled by controller 52.

In order to observe the individual wells or holes of the cell carrier 2, one surface of the cell carrier is illuminated with a source 13, and a collimator 15 may optionally be disposed between the source 13 and the cell carrier 2. An observation system 10, such as an Olympus BX61 motorized research microscope, available from Olympus America Inc., of Melville, N.Y., USA, may be disposed at the opposite surface of the cell carrier, in order to view the illumination from the source 13 passing through the cell carrier 2. Observation system 10 may include an adjustable focus lens 12 and a detection array 14 of a plurality of light responsive elements 16. In the example shown in FIG. 1, a charge-coupled device (CCD) array of a digital camera, such as the DP70, also available from Olympus America Inc., may be used to convert illumination impinging on detection array 14 into electronic signals. Adjustable focusing lens 12 may be functionally associated with a focusing motor 18 controlled by a focus controller 20. Observation system 10 may be functionally associated with a focus control component 20 and an image processing unit 22, which in the exemplary system of FIG. 1 is shown as a computer 24 configured with hardware and software to manipulate electronic signals received from detection array 14 as an image, and to process the individual pixels of the image as desired. Commercially available software suitable for such image processing is, for example, Image Pro Plus, available from Media Cybernics Inc., of Silver Spring, Md., USA.

The control computer 24 functionally associated with focus control component 20 and image processing component 22, may also provide control inputs to motor controller 52. In variations of what is shown in FIG. 1, and/or focus control component 20 and/or image processing component 22 and/or motor controller 52 may be incorporated in computer 24, for example as software running on control computer 24, so that physically computer 24 may be the only control component present, and the functions of controllers/control components 9, 20, 22 and 52 may all be effected by computer 24. Similarly, control of the illumination source 13 may also be effected by computer 24.

The illumination source generally used in such systems are of visible or ultraviolet light, depending on the nature of the cells to be observed, although it will be appreciated that in some circumstances it may be desired to use an illumination source emitting at other wavelengths. The range of wavelengths over which each source will illuminate may be chosen in accordance with the particular application. In some implementations, illumination source 13 may be a source of visible light.

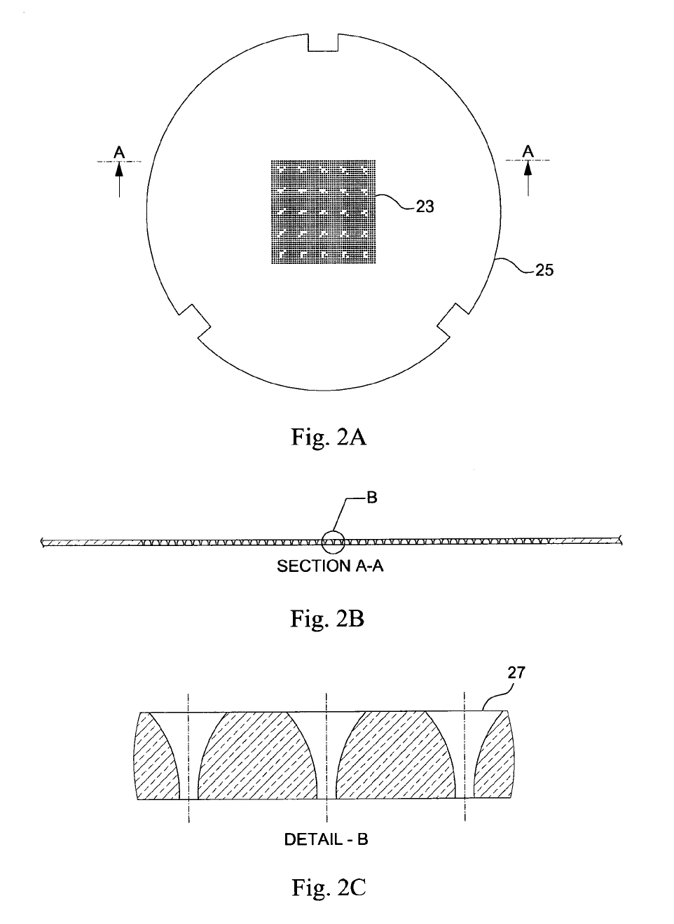

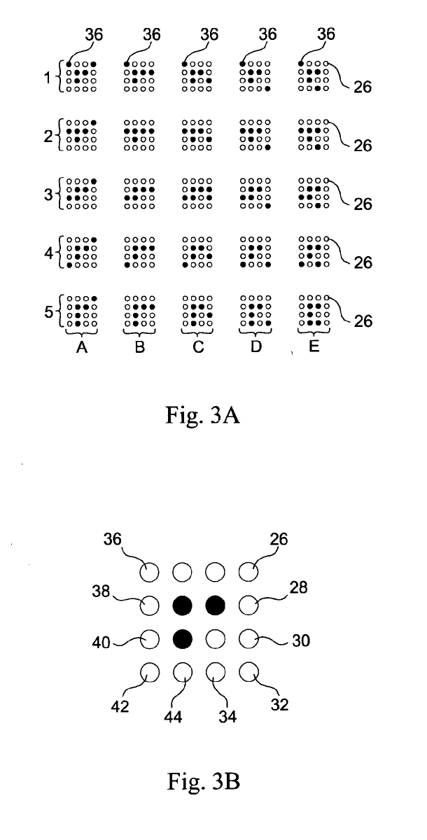

FIG. 2A shows a plan view of one example of a hole-type cell carrier 23 whose construction and operation is described in this disclosure. In the example of FIG. 2A, the carrier 23 is essentially a grid formed or mounted in the middle of a circular disk 25, which may conveniently be of approximately 6 mm diameter. The disk 25 is notched to ensure proper alignment on a cell carrier holder (not shown in the disclosure). The grid may have 2500 individual cell-capturing depression locations; at most of these locations are located holes. As will be explained in more detail with reference to the detailed example of FIGS. 3A and 3B below, the 2500 individual cell-capturing depression locations may be divided into 25 regions of 100 individual cell-capturing depression locations each, and combinations of holes from among the central 16 cell-capturing locations out of the 100 locations in each region, function as indicia to indicate the relative orientation and identity of each region. It is to be understood that the field of view of the cell viewing microscope, or of whatever device is used to view the cell carrier, should be sufficiently large, even at usefully high magnification levels, that the viewing device will always image at least one of the elements of the central 16 cell-capturing locations of at least one region, so that the system controller has at least one set of location/orientation defining data on which to commence acquisition of the location/orientation data at procedure start-up. Although all 16 of the locations are needed in order to fully define the location/orientation of the region, as will be explained below, there may also be practical applications for a system having region spacing and device magnification in which even only a single coding element appears in the image, since even if a single coding element is visible, the user, or an automatic image processing system, can detect this single element and then knows how to move the image so that all of the coding elements of that region become visible, to fully define the location and orientation. In FIG. 2A, the dark spots represent individual cell-capturing depression locations that are transparent (i.e. holes), whereas white spots at individual cell-capturing depression locations represent locations that have been rendered to be substantially not transparent. As will be explained hereinbelow, the positions of those holes rendered non-transparent, are arranged according to a predetermined pattern, whose use will be explained hereinbelow.

FIG. 2B is a side view of the row of holes taken along line A-A in FIG. 2A.

FIG. 2C, which is an enlarged portion (corresponding to “B”) of the row of holes shown in FIG. 2B, is a schematic cross sectional view of representative holes, shaped and sized in this example to each hold a single living cell. The holes in this example have a large aperture at one face of approximately 20 microns diameter and a small aperture near the other face of approximately 5 microns diameter, for observing cells where those dimensions are suitable.

Reference is now made to FIG. 3A, which depicts schematically the central 16 i cell-capturing depression locations of 25 regions of an exemplary cell carrier, such as that shown in FIG. 2A, having 25 regions of cell-capturing depressions. It will be appreciated, however, that the scheme depicted in FIG. 3A may also be utilized with well-type cell carriers. In FIG. 2A, the cell carrier has 2500 cell-capturing depression locations, divided into 25 regions each having 100 cell-capturing depression locations. The 25 regions are arranged as 5 regions by 5 regions, each region having 10 rows of cell-capturing depression locations by 10 columns of cell-capturing depression locations, such that each region contains 100 locations, of which, only the central 16 locations are shown in the regions shown in FIG. 3A. However, it is to be understood that such a sized cell carrier is only an example used to describe the device of this application, and that the number of cell-capturing depression locations per region of the cell carrier which is represented in FIG. 3A may be greater or less than 100, and the number of regions may be greater or less than 25, and may be arranged differently from a square layout. In FIG. 3A, the dark spots are meant to represent cell-capturing depression locations that have been rendered opaque to the illumination used, whereas white spots at individual cell-capturing depression locations represent hole locations that are transparent to the illumination used.

As shown in the example of FIG. 3A, of the central 16 cell-capturing depression locations in each region, the central four cell-capturing depression locations of all 25 regions are identically arranged, and are utilized to indicate orientation: 3 of the 4 central cell-capturing depression locations being opaque to the illumination used. The three opaque cell-capturing depression locations of the central 4 cell-capturing depression locations in each region thus form a triangle in each region, each triangle pointing, in the example shown in FIG. 3A, toward the upper left-hand corner of the cell carrier. Although a triangular shape of three hole locations defines absolute direction in an unambiguous manner, it is also possible to use a predetermined convention for defining orientation, such as a single marked hole, or two marked holes in positions of the central 16 locations of the array, where the significance of the position or positions with regard to the orientation is predetermined.

Furthermore, in the example shown in FIG. 3A, 10 of the remaining 12 individual cell-capturing depression locations adjacent to and surrounding the central four cell-capturing depression locations are used to indicate the identity of the region, using combinations of cell-capturing depression locations that are optically substantially opaque or non-transmissive, and cell-capturing depression locations which allow greater optical transmission. As shown representatively in FIG. 3B, the cell-capturing depression location in the upper right-hand corner (using throughout this description, the spatial connotation of the page of the drawing) of each of the groups of 12 cell-capturing depression locations is numbered 26. The cell-capturing depression locations below location 26 are numbered, respectively, 28, 30 and 32. The cell-capturing depression location to the left of location 32 is numbered 34. As can be seen in FIG. 3A, when location 26 is opaque, or alternatively, when location 26 is not highly transparent, for example it is a hole that is filled with material that is opaque, and locations 28, 30, 32 and 34 are essentially transparent, or at least delectably more transparent to visible light than location 26, this indicates that the region is located in column A, i.e. the left-most column of five regions as shown in FIG. 3A. When location 28 is opaque or not highly transparent, and locations 26, 30, 32 and 34 are essentially transparent, this indicates that the region is located in column B. When location 30 is opaque or at least not highly transparent, and locations 26, 28, 32 and 34 are essentially transparent, this indicates that the region is located in column C, i.e. the middle column of five regions as shown in FIG. 3A. When location 32 is opaque or at least not highly transparent and locations 26, 28, 30 and 34 are essentially transparent, this indicates that the region is located in column D. When location 34 is opaque or at least not highly transparent and locations 26, 28, 30 and 32 are essentially transparent, this indicates that the region is located in column E, i.e. the right-most column of five regions as shown in FIG. 3A.

Similarly, the cell-capturing depression location in the upper left-hand corner of each group, as partially shown in FIG. 3A and as shown representatively in FIG. 3B, is numbered 36. The locations below this location, as shown in FIG. 3B, are numbered 38, 40 and 42 respectively. The location to the right of location 42 is 44. As shown in FIG. 3A, when location 36 is optically opaque or at least not highly transparent to visible light (for example it is a hole that is filled with material that is essentially opaque to visible light), and locations 38, 40, 42 and 44 are transparent to visible light (i.e. at least detectably more transparent to visible light than location 26), this indicates that the region is located in row 1, i.e. the uppermost row of five regions as shown in FIG. 3A. When location 38 is opaque or not highly transparent to visible light, and locations 36, 40, 42 and 44 are essentially transparent to visible light, this indicates that the region is located in row 2. When location 40 is opaque or at least not highly transparent to visible light, and locations 36, 38, 42 and 44 are essentially transparent to visible light, this indicates that the region is located in row 3, i.e. the middle row of five regions as shown in FIG. 3A. When location 42 (42′, etc.) is opaque or at least not highly transparent to visible light, and locations 36, 38, 40 and 44 are essentially transparent to visible light, this indicates that the region is located in row 4. When location 44 is opaque or at least not highly transparent to visible light, and locations 36, 38, 40 and 42 are essentially transparent to visible light, this indicates that the region is located in row 5 i.e. the bottommost row of the five regions as shown in FIG. 3A.

In this way, both the orientation of the cell carrier and the identity of each region being viewed can be determined, by irradiating illuminating the cell carrier, (or just the region of interest) with visible light below the cell carrier, detecting the pattern of light illumination transmitted therethrough, and using the this pattern of light transmitted therethrough to determine the orientation and identity of the specific region. Such a determination may be made manually or, using suitable computer software, automatically. Automatic determination of the orientation of the cell carrier and identification of each region enables the system to move the cell carrier to such that a particular position or positions are observed, for example if it is desired to repeatedly observe one or more living cells at particular locations over a period of time.

It will be appreciated that FIG. 3A depicts merely one embodiment example of this implementation, and that many variations on this embodiment are possible. For example, relative to the surrounding cell-capturing depression locations, the triangle formed by three of the four center cell-capturing depression locations could point to a different corner of the cell carrier. Similarly, a different combination of cell-capturing depression locations may be used to indicate the row and column in which the region is located.

A practical and cost effective method of producing cell carriers is by injection molding of a plastic material. For such a manufacturing method, the coding information can be incorporated in the mold used to produce the cell carrier. By this means, the cell carrier is produced in one manufacturing step with the coding information already implanted. For a closed well type of cell capturing depression made of an optically transparent material, the difference in optical transmission required to define a coding element can be produced, for instance, by making the bottom of the coding position well optically diffusive, such that the transmission of the light through the bottom of such a coded location is reduced. For a hole type of cell capturing depression, the coding can be generated by making blind holes at the coding positions, i.e. holes with a bottom, such that the decreased transmission due to the bottom material can be detected. Such a bottom can also be made diffusive to increase the transmission discrimination. Alternatively, if it is desire not to lose any coding positions by closing off their bottoms, a diffusive mark can be generated around the perimeter of the hole, or a distinguishing mark can be imprinted next to the hole on the top surface of the cell carrier, or any other distinctive marking method may be used for this purpose, the marking being detected and identified by the image processing unit of the system. For a cell carrier made of an opaque material, the coding information may be provided by generating the coding locations by means of small holes, such that the coding marks are essentially transparent. The coding holes must be sufficiently small that they do not interfere with the process of entry of the cells into the depressions, generally by suction.

As an alternative to generating the coding marks on the cell carrier during manufacture, the coding marks may be added following manufacture. This can be done by screen printing methods, or by any other method used to imprint marks on a product. If the cell carrier is transparent, then an opaque spot of ink or paint in the bottom of the well, or lodged in the hole if open, can be conveniently used as the coding medium.

The material used to fill the hole should be affixed in place. This can be achieved, for example, by polymerizing the non-transparent polymer in the well or the hole, or by dissolving previously polymerized material in a solvent, depositing the material at the appropriate location, removing the solvent, and, if necessary, melting (e.g. using a laser) or ultrasonically welding the non-transparent material to the hole.

According to other exemplary applications described in this disclosure, when the cell carrier is a hole-type carrier, it may be constructed from material which is itself opaque to the illumination used, and the holes at cell-capturing depression locations where cells will be captured when the cell carrier is used, are made to be transparent. In such cases, groups of cell-capturing depression locations that collectively serve to identify the orientation and the identity of the region, may be formed by refraining from forming holes at the appropriate particular locations. Thus, for example, if the holes in the hole-type cell carrier are formed by laser machining, the cell-capturing depression locations which are to be used to identify the orientation and identity of the region are not laser machined, thus leaving opaque material at those locations. Similarly, if photolithography or electroforming is used to make the hole-type cell carrier from non-transparent material, the photomask used in the process may be designed so that when the process is complete, holes are only formed at cell-capturing depression locations where illumination is to pass through, and holes are not formed at the cell-capturing depression locations used to identify the orientation and identity of the region.

It will also be appreciated that in FIG. 3A, only 10 of the 12 cell-capturing depression locations surrounding the central four cell-capturing depression locations in each region are utilized to indicate the identity of the region. By utilizing all 12 of the cell-capturing depression locations surrounding the central 4 cell-capturing depression locations in each region to indicate the identity of the region, the number of regions could be expanded from 25 to 36, and the total numbers of cell-capturing depression locations in the grid could be expanded from 2500 to 3600-6 cell-capturing depression locations to indicate the column by 6 cell-capturing depression locations to indicate the row totaling 36 regions of 100 cell-capturing depression locations each.

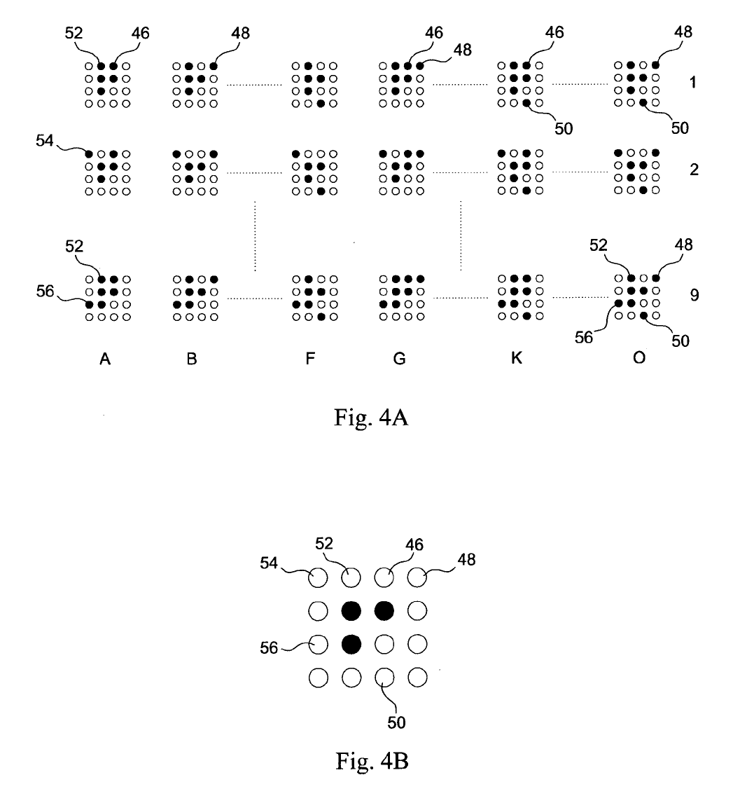

Furthermore, by using combinations of 2 cell-capturing depression locations to identify the row (out of the 6 cell-capturing depression locations available for this purpose) and 2 cell-capturing depression locations to identify the column (out of the 6 cell-capturing depression locations available for this purpose), the number of regions can be increased by 225 (15 additional combinations of row identifiers and 15 additional combinations of column identifiers). This is illustrated schematically in FIGS. 4A and 4B, in which opaque location 46 alone indicates that the region is located in column A, opaque location 48 alone indicates that the region is located in column B, and opaque location 50 alone indicates that the region is in column F, but the combination of opaque locations 46 and 48 indicates that the region is located in column G, the combination of opaque locations 46 and 50 indicates that the region is in column K, and the combination of opaque locations 48 and 50 indicates that the region is in column 0. Similarly, when location 52 alone is opaque, this indicates that the region is in row 1, when location 54 alone is opaque, this indicates that the region is row 2; when locations 52 and 56 are both opaque, this indicates the region is row 3. Of course, if it is desired to use a smaller number of regions, this is also possible, in which case fewer of the 12 cell-capturing depression locations surrounding the central 4 cell-capturing depression locations would be utilized. Furthermore, it will be appreciated that the size of each region may be chosen to be different from that shown in FIGS. 2A to 4B, e.g. 36, 49, 64, 81, 121, 144, 169, 196, 225, 256, 289, 324, 361 or 400 wells/holes per region, and that the number of cell-capturing depression locations used to indicate the orientation of the region or the identity of the region may be other than the number shown in FIGS. 2A to 4B.

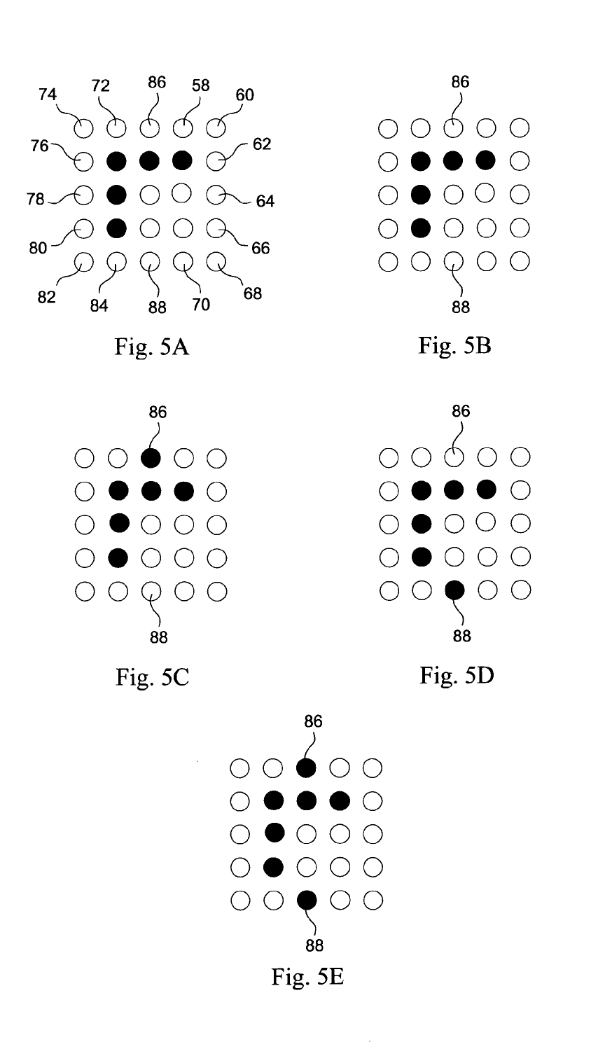

It will also be appreciated that instead of utilizing 16 cell-capturing depression locations to identify the orientation and identity of a region, as shown in FIGS. 3A to 4B, a different number of cell-capturing depression locations may be used for this purpose. Thus, for example, FIG. 5A, which is analogous to FIGS. 3B and 4B, illustrates schematically how a group of 25 cell-capturing depression locations may be used to identify the orientation and identity of a region. The five darkened spots (out of the central 9 spots) represent cell-capturing depression locations that are opaque. As shown in FIG. 5A, these form a triangle, which points toward the upper left-hand corner of the cell carrier (analogously to the central three darkened spots of FIGS. 3B and 4B). However, unlike FIGS. 3B and 4B, the number of cell-capturing depression locations along the perimeter of the central group used to indicate rows and columns is now 14 (out of the total of 16 locations around the perimeter) rather than 12, thus facilitating identification of up to 49 regions (7 rows×7 columns) if single locations are used to indicate rows and columns respectively. Thus, the locations marked 58, 60, 62, 64, 66, 68 and 70 may indicate that the region is located in a specific one of 7 columns, and the locations marked 72, 74, 76, 78, 80, 82 and 84 indicate that the region is located in a specific one of 7 rows.

It is to be understood that the symmetrical arrangement of the central depressions containing the coding locations shown in the regions of FIGS. 3A to 5E are only exemplary arrangements, and that it is possible to use other, less symmetrical arrangements also, such as 3×5 or 4×5 depressions. Furthermore, the set of depressions containing the coding locations need not be arranged rectilinearly, but could be arranged in any other predetermined shape.

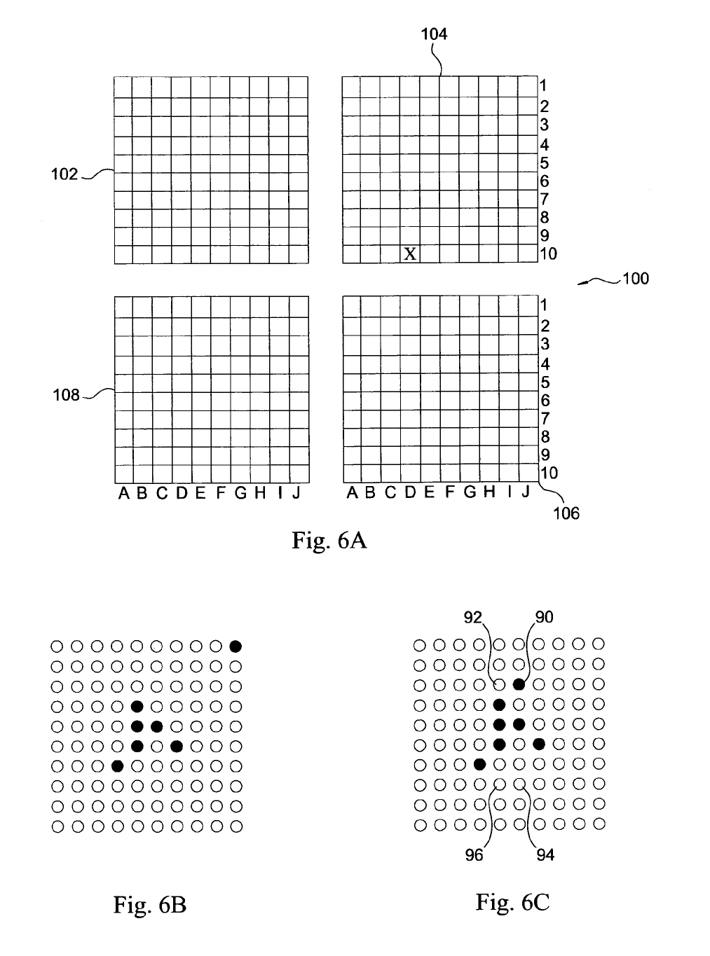

In some implementations, the locations marked 86 and 88 may be utilized to indicate an eight row/column arrangement, thus facilitating identification of up to 64 regions. In other examples, in which two to four grids are included on a single cell carrier, locations 86 and 88 may be used to indicate in which of the two to four grids the region is located, as will be explained in more detail below, in connection with the examples of FIGS. 6A to 6D. It will be appreciated that in FIG. 5A, as in FIGS. 5B to 5E, for the sake of simplicity none of locations 58, 60, 62, 64, 66, 68, 70, 72, 74, 76, 78, 80, 82 or 84 are darkened.

Reference is now made to FIG. 6A, which depicts schematically an example of a cell carrier 100 containing four grids, 102, 104, 106, 108, each containing 10,000 cell-capturing depression locations (100 rows×100 columns). As shown by the dotted lines in FIG. 6A, each grid is subdivided into 100 regions, each region containing 100 locations arranged in a 10 row by 10 column layout, as illustrated by each of exemplary FIGS. 6B to 6D. As shown representatively in FIG. 6B, which could correspond to the region marked with an “X” in FIG. 6A, the central 16 cell-capturing depression locations in each region are used to identify the orientation and identity of the region, in the manner shown in FIG. 4A; in the case of FIG. 6B, the region is located in column D, row 10, as marked in FIG. 6A by the “X”. However, unlike the example of FIG. 4A, in FIG. 6B one of the cell-capturing depression locations, such as in the corner of each region, may be utilized to identify the grid in which the region is located. Thus, for example, when the upper right cell-capturing depression location (relative to the direction of the triangle formed in the center of the region) is opaque, as shown in FIG. 6B, this may indicate that the region is located in the upper right grid 104 of the cell carrier 100; when the upper left cell-capturing depression location is opaque, this could indicate that the region is located in the upper left grid 102; when the lower right cell-capturing depression location is opaque, this may indicate that the region is located in the lower right grid 106; when the lower left cell-capturing depression location is opaque, this could indicate that the region is located in the lower left grid 108.

Alternatively, as shown representatively in FIGS. 6C and 6D, one of the locations, such as those numbered 90, 92, 94 and 96 may be used to indicate the grid in which the region is located, opaque location 90 indicating for instance, that the region is in the upper left-hand grid 102, opaque location 92 indicating that the region is in the upper right-hand grid 104, opaque location 94 indicating that the region is in the lower left-hand grid 108, and opaque location 96 indicating that the region is in the lower right-hand grid 106. In practice, such an arrangement may be more efficient in some respects that the arrangement shown in FIG. 6B, since the location used to indicate the grid in which the region is located is adjacent to the locations used to indicate the identity and orientation of the region, thus facilitating quicker identification by an optical system, which needs to scan a more compact area to acquire the information needed to define location.

Similarly, referring back to FIGS. 5B to 5E, if the central 25 cell-capturing depression locations are utilized to indicate the orientation and identity of the region, when a plurality of grids are employed, locations not used for defining position or orientation within the region, such as 86 and 88, may be used to indicate the grid in which the region is located: when neither cell-capturing depression location 86 nor 88 is opaque to the applied illumination (FIG. 5B), this indicates that the region is located in the upper left grid 102; when only cell-capturing depression location 86 is opaque (FIG. 5C), this indicates that the region is located in the upper right grid 104; when only cell-capturing depression location 88 is opaque (FIG. 5D), this indicates that the region is located in the lower left grid 108; and when both cell-capturing depression locations 86 and 88 are opaque (FIG. 5E), this indicates that the region is located in the lower right grid 106.

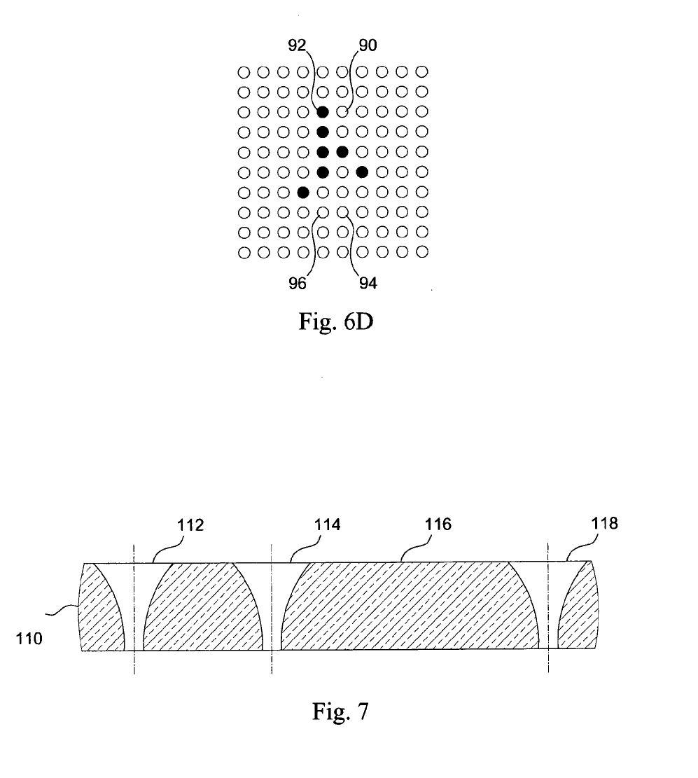

Reference is now made to FIG. 7, which illustrates schematically a cross sectional view of another exemplary cell carrier 110, in which the indicia marking at the relevant cell capturing location is achieved by the omission of the cell capturing depression itself. Thus, in FIG. 7, which is a cross sectional view of a row of such depressions, 112, 114, 118, the depression which should have been situated at the location 116 between depressions 114 and 118 is missing. When viewed by the observation system, even though the cell carrier material itself may be nominally transparent, there will be detected a difference in transmission between the holes of the depressions 112, 114, 118, and the transmission in the region 116 of the missing depression. The difference in transmission will not be as great as that of the previous examples, where a situation closer to a true transparent/opaque relationship may be engendered, but the optical viewing system programs should be designed such that this difference in transmitted illumination should be readily detectable. This implementation thus enables the coding to be performed at the time of manufacture of the cell carrier itself, without the need of the added step of generating the opaque mark on or at the depression location to be marked.

It is to be understood that throughout this disclosure, the terms transparent and opaque are meant to relate to the comparative transmission of the locations being referred to, and not to absolute transmission levels. Thus, a marked depression location which is designed to transmit noticeably less illumination than the surrounding cell carrier material is understood to be “opaque” in comparison to the “transparent” nature of the surrounding material, and coding indicia may thus be formed in that manner too.

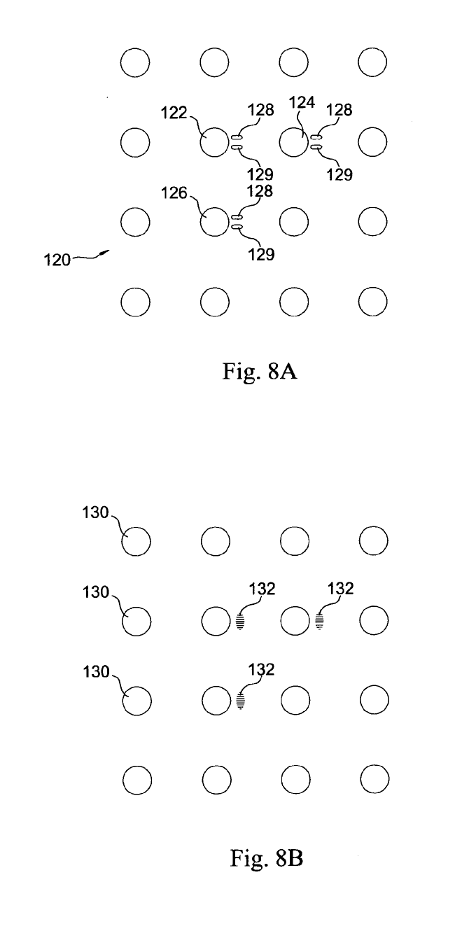

In the previously described examples of cell carriers, the indicia have been implemented by differences in transmission between the marked depression location and the surrounding cell carrier and surrounding unmarked depressions. However, any other suitable method of marking the cell-capturing depression to act as indicia may also be used. One such method is by modifying the illumination passing through the location of the desired indicia by means of an optical interference effect. This is illustrated in FIG. 8A, where there is shown a plan view of the section of an exemplary region of a cell carrier 120, where the indicia markings are to be applied. The section contains 16 cell capturing depression, including, as an example, the three orientation defining indicia shown in FIG. 3B. At cell capturing depressions 122, 124 and 126, which are intended to be used as indicia location, pairs of openings 128, 129 are provided, the openings being sufficiently narrow and sufficiently close together relative to the wavelength of the light used, that an interference phenomenon is generated by the light passing through them. The openings 128, 129 shown in FIG. 8A are elongated openings, almost in the form of slits, such that a series of interference fringes parallel to the long axis of the openings should be formed in the imaging plane of the viewing system, located along the perpendicular to the drawing plane. A typical image pattern that could be obtained in the imaging system from the arrangement of indicia shown in FIG. 8A is shown in FIG. 8B. The illumination obtained from unmarked cell capturing depressions will be generally uniform 130, while that from the indicia marked depression locations will show a characteristic fringe pattern 132, with the fringe spacing being dependent on the wavelength of light used, the distance apart of the openings 128, 129, and the distance between the exit plane of the illumination passing through the cell carrier, and the effective focal plane of the imaging system used. The image processing program may be adapted to recognize the presence of the rapidly changing intensity profile associated with the fringes, and to determine that a marker index is located at that position. In general, the light used in such a cell observation system is monochromatic, since it is desired to excite fluorescence in the captured cells, and to filter out the incident light from the fluorescent light. Therefore, high contrast fringes should be achievable. However, since the source used is not generally a coherent source, the fringe contrast may be somewhat diminished by the lack of good coherence between the incident light on the two openings. It is to be understood that the form of the openings shown in the example of FIG. 8A, and their location relative to the cell capturing depression are only examples of this interference implementation for providing the indices for such cell carriers, and that the device is not intended to be limited by the specific parameters and positions shown in FIG. 8A. Thus, for instance, alternative use of a single small hole next to the marked cell capturing depression would result in diffraction rings being formed on the image plane.

The essence of the methods and devices presented in the present disclosure revolve around the concept of regional coding, and the various implementations of these ideas are intended to be only examples thereof, and are not intended to limit the scope of the disclosure, nor are they intended to be considered as the only implementations possible for carrying out the invention. Thus for instance, the above examples of carrier cell coding arrangements have generally been described in terms of cell carriers in which light is transmitted through the cell carrier from one surface to the opposite surface, and the perturbation of this transmission at the coding marks is detected in a transmission image. However, it is to be understood that the coding methods and devices described in this disclosure are not meant to be limited to transmissive implementations only, but can equally well be applied using other means of detecting the coding locations.

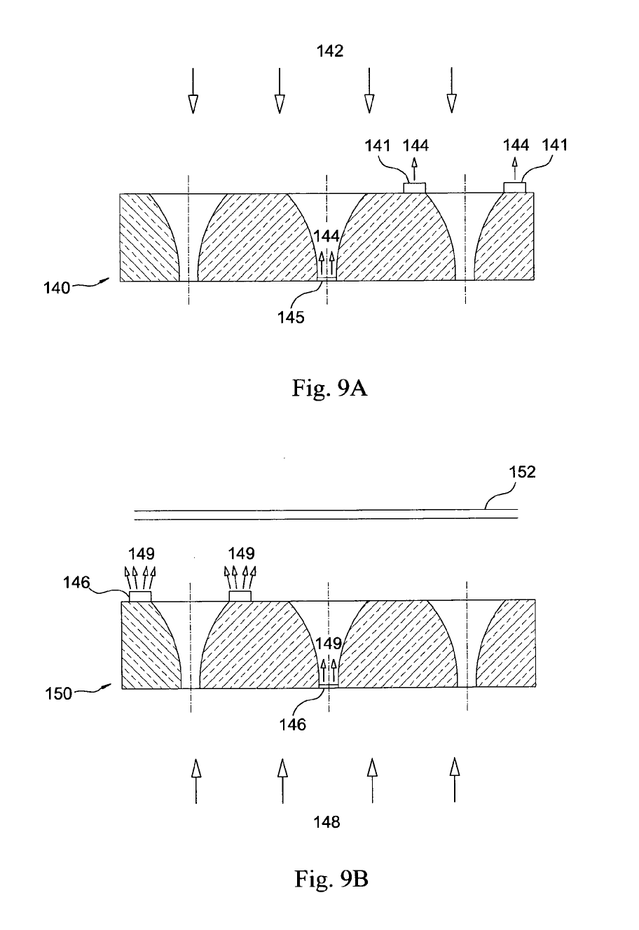

Reference is thus made to FIGS. 9A and 9B, which illustrate schematically other methods by which the coding markings can be detected. In FIG. 9A, there is shown a cross section of a cell carrier 140 having reflective marks at the coding locations, wherein the illumination of the coding markings and its detection are performed from one side of the cell carrier. The markings are in the form of reflective spots or areas, which can be located either in the cell capturing depressions themselves, as in that marked 145, or on the top surface of the cell carrier close to the cell capturing depression, as in that marked 141. When illuminated, the marks reflect light back 144 in the direction of the illumination. An imaging device can then detect this reflected light and is able to map the location marker locations accordingly.

In FIG. 9B, there is shown a cross section of a cell carrier 150 having fluorescent or phosphorescent marks 146 at the coding locations. The illumination 148 directed at the cell carrier has a wavelength which is selected to excite a fluorescent or phosphorescent emission 149 from the marking spots 146. As with the embodiment of FIG. 9A, the fluorescent or phosphorescent marker material can be located anywhere which associates it uniquely with the coding location to be marked, and where the exciting illumination 148 does impinge on it. A filter 152, transmitting essentially only the fluorescence or phosphorescence emission, can be used to increase the detection discrimination abilities of the emitting coding markers. The exciting illumination can be directed at the cell carrier either from the side opposite to that of the viewing or imaging device, as shown in FIG. 9B, or from the same side as the viewing or imaging device. The illumination source may be the same one as is used for the cell fluorescence inspection, or it may be at a different wavelength.

It will be appreciated that the invention is not limited by the examples shown in the figures, and that other variations will be readily apparent to those skilled in the art upon reading this description.

1. A cell carrier comprising a plurality of cell-capturing depressions organized into a plurality of regions, some of the cell-capturing depressions of each of said regions being marked, wherein the spatial arrangement of said marked cell-capturing depressions in a region identifies the location of said region within the cell carrier.

2. A cell carrier according to claim 1, wherein said spatial arrangement of said marked cell-capturing depressions within a region further provides an indication of the orientation of that region.

3. A cell carrier according to claim 1, wherein said marked cell-capturing depressions are arranged in predetermined locations in a section of said regions, such that said location of said region within the cell carrier is identified by inspection of said predetermined locations in said section of said region.

4. A cell carrier according to claim 3, wherein said predetermined locations are arranged such that at least one marked depression is visible in an image of said region taken at a magnification significantly higher than that which enables the entire cell carrier to be imaged.

5. A cell carrier according to claim 3, wherein said predetermined locations are arranged such that all of said marked cell-capturing depressions of a specific region are visible in an image of said region taken at a magnification significantly higher than that which enables the entire cell carrier to be imaged.

6. A cell carrier according to claim 1, wherein the marks of said marked cell-capturing depressions possess a detectable property that distinguishes a marked cell-capturing depression from other cell-capturing depressions in the cell carrier.

7. A cell carrier according to claim 6, wherein said detectable property is selected from the group consisting of (a) transmission of electromagnetic radiation of a particular wavelength or range of wavelengths; (b) absorption of electromagnetic radiation of a particular wavelength or range of wavelengths; (c) fluorescence at a particular wavelength in response to stimulation at a different particular wavelength; (d) reflection of electromagnetic radiation of a particular wavelength or range of wavelengths; and (e) optical interference of electromagnetic radiation of a particular wavelength or range of wavelengths.

8. A cell carrier according to claim 6, wherein said detectable property is absorption of electromagnetic radiation in the visible spectrum, such that said marking has decreased optical transmission in comparison with other areas of said cell carrier.

9. A cell carrier according to claim 1, wherein said cell carrier is constructed of a material which is substantially transparent to visible light, and said marking is substantially opaque to visible light.

10. A cell carrier according to claim 1, wherein said marking of a cell-capturing depression is located either within said cell-capturing depression or in the vicinity of said cell-capturing depression.

11. (canceled)

12. A cell carrier according to claim 3, wherein said section of said regions containing the marked cell-capturing depressions comprises a first set of marked cell-capturing depressions and a second set of unmarked cell-capturing depressions.

13. A cell carrier according to claim 12, wherein said first set of marked cell-capturing depressions are associated with four cell-capturing depression locations arranged in a quadrilateral pattern.

14. A cell carrier according to claim 12, wherein the location of some of said first set of marked cell-capturing depressions defines the spatial orientation of said region, and at least one other of said first set of marked cell-capturing depressions defines the location of said region in said cell carrier.

15. A method for identifying the location of a region of a cell carrier under observation, comprising:
providing a cell carrier comprising a plurality of cell-capturing depressions organized into a plurality of regions, some of the cell-capturing depressions of each of said regions being marked, the spatial arrangement of said marked cell-capturing depressions in a region identifying the location of that region within the cell carrier;
illuminating said cell carrier with a source;
detecting marked cell-capturing depressions in an image of said cell carrier; and
identifying the location of said region of said cell carrier from said spatial arrangement of said marked cell-capturing depressions.

16. A method according to claim 15, wherein said method also comprises the step of detecting the orientation of said region from said spatial arrangement of said marked cell-capturing depressions.

17. A method according to claim 15, wherein said image of said cell carrier is obtained at a magnification significantly higher than that which enables the entire cell carrier to be imaged.

18. A method according to claim 15, wherein said method enables the identification of the location of said region without reduction in the magnification used to view cell capturing depressions in said cell carrier.

19. A method for identifying the location of a specific cell capturing depression under observation in a cell carrier, comprising the steps of identifying the region of said cell carrier in which said specific cell capturing depression is located according to claim 15, and determining the location of said specific cell capturing depression within said region.

20. A method according to claim 19, further comprising the optional step of detecting the orientation of said region of said cell carrier in which said specific cell capturing depression is located before determining the location of said specific cell capturing depression within said region.

21. A method according to claim 19, wherein said step of determining the location of said specific cell capturing depression within said region is performed by an image processing routine using said spatial arrangement of marked cell-capturing depressions and the known extent of a region around said spatial arrangement of marked cell-capturing depressions.

22. A method according to claim 15, wherein said method enables the return to the observation of a previously viewed cell in a cell-capturing depression without the need to search for location identifying information at low magnification.

23. A method according to claim 15, wherein the marking of said marked cell-capturing depressions possess a detectable property that distinguishes a marked cell-capturing depression from other cell-capturing depressions in the cell carrier.

24. A method according to claim 23, wherein said detectable property is selected from the group consisting of (a) transmission of electromagnetic radiation of a particular wavelength or range of wavelengths; (b) absorption of electromagnetic radiation of a particular wavelength or range of wavelengths; (c) fluorescence at a particular wavelength in response to stimulation at a different particular wavelength; (d) reflection of electromagnetic radiation of a particular wavelength or range of wavelengths; and (e) optical interference of electromagnetic radiation of a particular wavelength or range of wavelengths.

25. A method according to claim 23, wherein said detectable property is absorption of electromagnetic radiation in the visible spectrum, such that said marking has decreased transmission in comparison with other areas of said cell carrier.

26. A method according to claim 15, wherein said cell carrier is constructed of a material which is substantially transparent to visible light, and said marking is substantially opaque to visible light.

27. A method according to claim 15, wherein said marking of a cell-capturing depression is located either within said cell-capturing depression or in the vicinity of said cell-capturing depression.

28. (canceled)
